(19) United States
(12) Patent Application Publication
(10) Pub. No.: US 2007/0027539 A1
(43) Pub. Date: Feb. 1, 2007

(54) Intaocular lens for inhibiting pco and aco

(76) Inventor: Joel Pynson, Toulouse (FR)

Correspondence Address: Bausch & Lomb Incorporated, One Bausch & Lomb Place, Rochester, NY, 14604-2701, US

(21) Appl. No.: 10/571,396

(22) PCT Filed: Sep. 30, 2003
(86) PCT No.: PCT/IB03/04703

§ 371 Date: Mar. 10, 2006

(51) Int. Cl. A61F 2/16

(52) U.S. Cl. 623/6.16; 623/6.46

(57) ABSTRACT

An intraocular lens for inhibiting posterior and anterior capsular opacification, or secondary cataract, includes an optic having a periphery provided with sharp bevels extending along arc segments defined by the juncture of the haptic-optic points of attachment and adjacent both the anterior and posterior optic surfaces.

### BACKGROUND OF THE INVENTION

The present invention relates to intraocular lenses (IOLs) for implantation in an aphakic eye where the natural lens has been removed due to damage or disease (e.g., a cataractous lens). The present invention more particularly relates to a novel IOL designed to inhibit the unwanted growth of lens epithelial cells (LECs) between the IOL and capsular bag and also on the IOL optic surface. When cell growth occurs along the posterior capsular wall, it is known as posterior capsule opacification or “PCO” to those skilled in the art. When cell growth occurs along the anterior capsular wall and anterior optic surface, it is known as anterior capsular opacification, or “ACO” to those skilled in the art.

A common and desirable method of treating a cataract eye is to remove the clouded, natural lens and replace it with an artificial IOL in a surgical procedure known as cataract extraction. In the extracapsular extraction method, the natural lens is removed from the capsular bag while leaving the posterior part of the capsular bag (and preferably at least part of the anterior part of the capsular bag) in place within the eye. In this instance, the capsular bag remains anchored to the eye's ciliary body through the zonular fibers. In an alternate procedure known as intracapsular extraction, both the lens and capsular bag are removed in their entirety by severing the zonular fibers and replaced with an IOL which must be anchored within the eye absent the capsular bag. The intracapsular extraction method is considered less attractive as compared to the extracapsular extraction method since in the extracapsular method, the capsular bag remains attached to the eye's ciliary body and thus provides a natural centering and locating means for the IOL within the eye. The capsular bag also continues its function of providing a natural barrier between the aqueous humor at the front of the eye and the vitreous humor at the rear of the eye.

One known problem with extracapsular cataract extraction is posterior capsule opacification, or secondary cataract, where proliferation and migration of lens epithelial cells occur along the posterior capsule behind the IOL posterior surface which creates an opacification of the capsule along the optical axis. This requires subsequent surgery, such as an Er:YAG laser capsulotomy, to open the posterior capsule and thereby clear the optical axis. Undesirable complications may follow the capsulotomy. For example, since the posterior capsule provides a natural barrier between the back of the eye vitreous humor and front of the eye aqueous humor, removal of the posterior capsule allows the vitreous humor to migrate into the aqueous humor which can result in serious, sight-threatening complications. It is therefore highly desirable to prevent posterior capsule opacification in the first place and thereby obviate the need for a subsequent posterior capsulotomy.

Various methods have been proposed in the art to prevent or at least minimize PCO and thus also the number of Er:YAG laser capsultomies required as a result of PCO. These PCO prevention methods include two main categories: mechanical means and pharmaceutical means.

In the mechanical means category of PCO prevention, efforts have been directed at creating a sharp, discontinuous bend in the posterior capsule wall which is widely recognized by those skilled in the art as an effective method for minimizing PCO. See, for example, Posterior Capsule Opacification by Nishi, Journal of Cataract & Refractive Surgery, Vol. 25, Jan. 1999. This discontinuous bend in the posterior capsule wall can be created using an IOL having a posterior edge which forms a sharp edge with the peripheral wall of the IOL.

By far the most promising method for inhibiting LEC formation on the posterior surface of an IOL is the mechanical means, i.e., by designing the IOL to have a sharp peripheral edge particularly at the posterior surface—peripheral edge juncture to create a discontinuous bend in the posterior capsule wall. This discontinuous bend in the posterior capsule wall has been clinically proven to inhibit the growth and migration of LECs past this bend and along the IOL surface. One of the early reports of this PCO-inhibiting effect of a planoconvex IOL may be found in Explanation of Endocapsule Posterior Chamber Lens After Spontaneous Posterior Dislocation by Nishi et al, J Cataract & Refractive Surgery-Vol 22, Mar. 1996 at page 273 wherein the authors examined an explanated planoconvex PMMA IOL where the posterior surface of the IOL was planar and formed a square edge with the peripheral edge of the IOL:

“Macroscopic view of the explanted IOL and capsule revealed a 9.5 mm capsule diameter. The open circular loops fit well along the capsule equator. The capsule equator not in contact with the haptic was also well maintained (FIG. 3). An opaque lens mass (Soemmering's ring cataract) was seen between the haptics and optic. The posterior capsule facing the IOL optic was clear.

Histopathological examination of the explanted capsule revealed few epithelial cells (LECs) on the posterior capsule. Between the loops and the optic, a lens mass with accumulation at the edge of the optic was seen (FIG. 4). There was an obvious bend in the posterior capsule at this site.” (Emphasis added.)

Thus, in the years since this report, the industry has seen much activity on creating IOLs with sharp posterior edges so as to create a sharp, discontinuous bend in the posterior capsule wall. While IOLs having a sharp posterior edge have proven to inhibit PCO compared to IOLs having rounded edges at the posterior surface-peripheral edge juncture, there still remains the possibility of LECs migrating along the posterior capsule and behind the IOL surface, especially if there is uneven contact and force of the IOL periphery with the capsular bag. This may happen, for example, should the IOL move within the capsular bag following surgery.

Besides the widely-known and discussed problem of PCO along the posterior capsular wall, anterior capsular opacification (ACO) may also occur when LECs migrate along the fragmented anterior capsule and continue to migrate along the anterior surface of the IOL optic. This problem is particularly prevalent in IOLs made of hydrophilic materials.

There therefore remains a need for an improved IOL design which addresses the problem of LEC migration along both the posterior and s anterior capsule and subsequent PCO and ACO formation, respectively.

### SUMMARY OF THE INVENTION

The present invention addresses the problem of both PCO formation and ACO formation by providing an IOL having a periphery including sharp edges along both the posterior and anterior peripheral edges of the optic body. The sharp edges extend the full circumference of the IOL body including the areas of haptic attachment to the optic. The sharp edge may be formed in the shape of a bevel having a pointed apex about the circumference of the optic as well as in the areas of haptic attachment. This configuration of the periphery of the IOL optic is a significant improvement over prior optic designs in that it provides improved barriers against LEC migration both posteriorly and anteriorly of the IOL optic. The optic periphery design is also relatively easy to manufacture compared with other, more complicated IOL periphery designs which have been proposed in the prior art for inhibiting LEC migration. See, for example, the following patents and publications which show various IOL optic periphery designs:
U.S. Pat. No. 5,171,320 issued to Nishi on Dec. 15, 1992
U.S. Pat. No. 5,693,093 issued to Woffmden et al on Dec. 2, 1997
U.S. Pat. No. 6,162,249 issued to Deacon et al on Dec. 19, 2000

### BRIEF DESCRIPTION OF THE DRAWING

FIG. 1 is a cross-sectional view of a human eye showing the natural lens within the capsular bag of the eye;

FIG. 2 is a cross-sectional view of a human eye showing the natural lens removed and replaced with a prior art IOL;

FIG. 3 is a perspective view of an IOL according to one embodiment of the invention;

FIG. 4 is a side elevational view thereof; and

FIG. 5 is an enlarged, fragmented, cross-sectional view showing the detail of the peripheral wall configuration of the IOL of the present invention.

### DETAILED DESCRIPTION

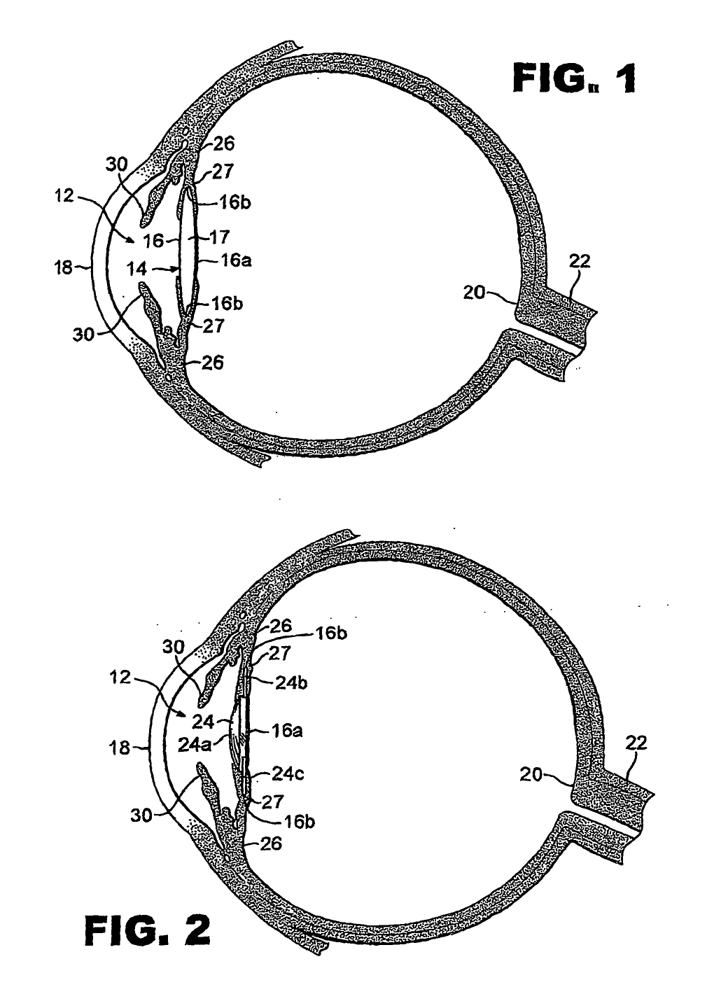

Referring now to the drawing, there is seen in FIG. 1 a cross-sectional view of a human eye 10 having an anterior chamber 12 and a posterior chamber 14 separated by the iris 30. Within the posterior chamber 14 is a capsule 16 which holds the eye's natural crystalline lens 17. Light enters the eye by passing through the cornea 18 to the crystalline lens 17 which act together to direct and focus the light upon the retina 20 located at the back of the eye. The retina connects to the optic nerve 22 which transmits the image received by the retina to the brain for interpretation of the image.

In an eye where the natural crystalline lens has been damaged (e.g., clouded by cataracts), the natural lens is no longer able to properly focus and direct incoming light to the retina and images become blurred. A well known surgical technique to remedy this situation involves removal of the damaged crystalline lens which may be replaced with an artificial lens known as an intraocular lens or IOL such as prior art IOL 24 seen in FIG. 2. Although there are many different IOL designs as well as many different options as to exact placement of an IOL within an eye, the present invention concerns itself with an IOL for implanting inside the substantially ovoid-shaped capsule 16 of eye 10. This implantation technique is commonly referred to in the art as the “in-the-bag” technique. In this surgical technique, a part of the anterior portion of the capsular bag is cut away (termed a “capsulorhexis”) while leaving the posterior capsule 16a intact and still secured to the ciliary body 26.

Thus, in the “in-the-bag” technique of IOL surgery, the IOL is placed inside the capsule 16 which is located behind the iris 30 in the posterior chamber 14 of the eye. An IOL includes a central optic portion 24a which simulates the extracted natural lens by directing and focusing light upon the retina, and further includes means for securing the optic in proper position within the capsular bag. A common IOL structure for securing the optic is called a haptic which is a resilient structure extending radially outwardly from the periphery of the optic. In a particularly common IOL design, two haptics 24b, 24c extend from opposite sides of the optic and curve to provide a biasing force against the inside of the capsule which secures the optic in the proper position within the capsule (see FIG. 2).

As stated in the Background section hereof, an undesirable post-surgical condition known as posterior capsule opacification or PCO may occur which results in an implanted IOL becoming clouded and thus no longer able to properly direct and focus light therethrough. The main cause for this condition is the mitosis and migration of lens epithelial cells (LECs) across the posterior surface of the capsule behind the IOL optic. As seen in FIG. 2, the posterior surface 16a of the capsule 16 touches the posterior surface of the IOL optic 24a. When the damaged natural lens is surgically removed, a number of LECs may remain within the capsule 16, particularly at the equator 16b thereof which is the principle source of germinal LECs. Although a surgeon may attempt to remove all LECs from the capsular bag at the time of IOL implantation surgery, it is nearly impossible to remove every single LEC. Any remaining LECs can multiply and migrate along the posterior capsule wall 16a. This is especially true in IOLs having rounded edges, where it has been found that clinically significant PCO results in about 20%-50% of patients three years post surgery. A presently popular and effective method of preventing PCO is to create a sharp, discontinuous bend in the posterior capsule wall 16a as explained in the Background section hereof.

In addition to the problem of PCO, another post-surgical condition known as ACO (anterior capsular opacification) may occur which also has the effect of obstructing clear vision. In this condition, LECs migrate along the remaining fragment of the anterior capsule and then migrate onto and along the anterior surface of the IOL optic. This occurs in IOLs made of hydrophilic materials, and especially hydrophilic materials having a water content in the range of about 18% to about 26%. Should this occur, the IOL may require explanting and implanting a new IOL into the eye, an undesirable prospect.

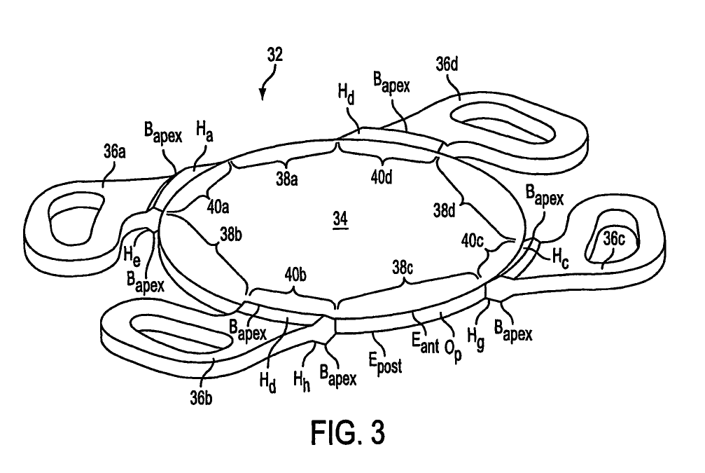

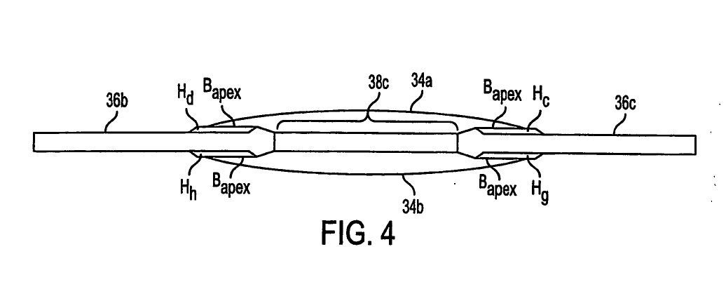

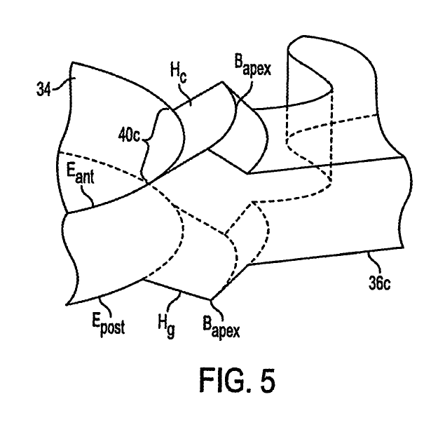

Referring now to FIGS. 3, 4 and 5, an exemplary embodiment of the inventive IOL 32 is shown. IOL 32 is seen to include a central optic portion 34 having opposite anterior and posterior surfaces 34a and 34b, respectively. When implanted within the eye, anterior optic surface 34a faces the cornea 18 and posterior optic surface 34b faces the retina 20. In this particular style of IOL, two pairs of haptics 36a, b and 36 c, d are attached to and extend from opposite sides of the periphery of optic portion 34 and are configured to provide a biasing force against the interior of the capsule 16 to properly position IOL 32 therein. More particularly, the haptics 36a-d are configured such that upon implanting the IOL with the capsular bag, the haptics engage the interior surface of the capsular bag. The engagement between the haptics and capsule creates a biasing force causing the IOL optic 34 to vault posteriorly toward the retina 20 whereupon the posterior surface 34b of the IOL optic presses tightly against the interior of the posterior capsule wall 16a of capsule 16. It is noted that the invention is not limited to the IOL style shown herein, but rather is useful in IOLs having any type and number of haptic elements. Furthermore, IOL 32 may be made from any suitable IOL material, e.g., PMMA, silicone, hydrogels and composites thereof, although it is particularly useful in IOLs made of the cell-loving materials described above. The IOL 32 may also be a one piece (formed from one single block of the same or dissimilar materials) or multiple piece design (e.g. where the haptics are attached to the optic after the optic is formed.)

Referring still to FIGS. 3, 4 and 5, it is seen that IOL optic 34 has an optic periphery Oₚ. For the purpose of description herein, arc segments of optic periphery Op will be respectively identified; particularly, those arc segments extending between the haptics (denoted by reference numerals 38a-d) interceded by those segments extending adjacent the haptics (denoted by reference numerals 40a-d).

The arc segments extending between the haptics 38a-d each include a sharp edge Eₐₙₜ defined adjacent the anterior optic surface 34a and a sharp edge Eₚₒₛₜ defined adjacent the posterior optic surface 34b. Edges Eₐₙₜ and Eₚₒₛₜ may be simply formed as shown in the drawing, i.e., by substantially right angles, or may assume any other suitable geometry to prevent LEC migration, e.g., those edge geometries shown herein with regard to the juncture of the haptics with the optic (described below) or as shown in commonly assigned U.S. Pat. No. 6,558,419. The sharp is edges Eₐₙₜ and Eₚₒₛₜ are effective at creating a bend in the anterior and posterior capsular wall along the associated arc segments 38a-d of the optic periphery when the IOL is implanted in the capsular bag as described above. Since the edges are provided adjacent both the anterior and posterior optic surfaces, LEC migration is likewise prevented along both the anterior and posterior bag walls along the associated arc segments 38a-d.

To prevent LEC migration along the remaining arc segments extending adjacent the point of haptic attachment to the optic periphery, sharp edges Ha-d are provided along arc segments 40a-d, respectively, adjacent anterior optic surface 34a, and sharp edges Hₑ₋ₕ are provided along arc segments 40a-d, respectively, adjacent posterior optic surface 34b. Sharp edges Hₐ₋ₕ may each be formed as a sharp bevel with the bevel apex Bₐₚₑₓ facing generally away from it's associated haptic element. It is understood, however, that the exact configuration of the sharp edges Hₐ₋ₕ may vary, the only requirement being that the edge acts to form a bend in the associated part of the capsular wall to prevent LEC migration past that point.

A presently preferred method of forming the multiple sharp edge configuration in the IOL optic 34 comprises a milling operation where the IOL optic is mounted to a fixture and a mill is used to cut into the posterior optic surface at the perimeter thereof. Other methods which may be employed to form the peripheral edge geometry include lathing and molding, for example. It is also preferred that IOL 32 undergo tumble polishing prior to forming the edge geometry so as to ensure the edges Eₐₙₜ, Eₚₒₛₜ, Hₐ₋ₕ, etc., retain their sharpness.

What is claimed is:

1. An intraocular lens for implanting in a human eye, comprising:
a lens optic having opposite anterior and posterior surfaces defined by an optic periphery;
one or more haptics attached to and extending from said optic periphery, the juncture of each said haptic and optic periphery defining an arc segment; and
characterized in that
first and second bevels each having a sharp apex formed along each said arc segment adjacent said anterior surface and said posterior surface, respectively.

2. The intraocular lens of claim 1, wherein said IOL optic is formed from a hydrophilic material.

3. The intraocular lens of claim 2, wherein said IOL is formed from a hydrophilic material having a water content in the range of about 18% to about 26%.

4. The intraocular lens of claim 1, wherein said optic periphery extending between said haptic-optic junctures includes a sharp edge Eₐₙₜ and Eₚₒₛₜ adjacent said anterior and posterior surfaces, respectively.

5. The intraocular lens of claim 1, wherein said lens optic and said one or more haptics are integrally formed together.

6. The intraocular lens of claim 1 wherein said lens optic and said one or more haptics are separately formed and subsequently attached together.
